(19) United States
(12) Patent Application Publication
(10) Pub. No.: US 2026/0286012 A1
(43) Pub. Date: Sep. 24, 2026

(54) ANTI-OUABAIN NANOBODY AND APPLICATION THEREOF

(71) Applicant: NANJING UNIVERSITY, Nanjing, Jiangsu (CN)

(72) Inventors: Wu Yin, Nanjing (CN); Chenming Xu, Nanjing (CN); Ping Wang, Jinan (CN); Huimin Zhang, Jinan (CN); Henda Li, Nanjing (CN); Tong Jin, Nanjing (CN)

(21) Appl. No.: 19/474,595

(22) PCT Filed: Jul. 29, 2024
(86) PCT No.: PCT/CN2024/108109

§ 371 Date: Oct. 10, 2025

(30) Foreign Application Priority Data
Aug. 2, 2023 (CN) 202310960079.1

(51) Int. Cl. C07K 16/44; C12N 1/20; C12N 15/70; C12R 1/19; G01N 33/543; G01N 33/94

(52) U.S. Cl. CPC C07K 16/44; C12N 1/20; C12N 15/70; G01N 33/543; G01N 33/9453; C07K 2317/14; C07K 2317/569; C07K 2319/21; C12R 2001/19

(57) ABSTRACT

The present invention belongs to the technical field of nanobodies, and more particularly, relates to an anti-ouabain nanobody and an application thereof. An amino acid sequence of the anti-ouabain nanobody disclosed by the present invention is as shown in SEQ ID NO. 1. A nucleotide sequence of the nanobody is shown in SEQ ID NO. 2. The nanobody has good affinity to ouabain, and can neutralize and antagonize the pharmacological activity of the ouabain, and detect the content of ouabain in a solution in combination with an ELISA method. The present invention provides a method for constructing the ouabain antibody with light weight, high thermal stability and high detection sensitivity; and provides a research tool for deeply excavating new biological functions of endogenous ouabain.

### CROSS REFERENCE TO RELATED APPLICATIONS

This is a National Stage of PCT International Application No. PCT/CN2024/108109, filed on Jul. 29, 2024, and this application claims priority of Application No. 202310960079.1 filed in China on Aug. 2, 2023, the entire contents of both of which are hereby incorporated by reference.

### REFERENCE TO AN ELECTRONIC SEQUENCE LISTING

The content of the electronic sequence listing (0168-0163PUS1-xml; Size: 14,688 bytes; and Date of Creation: May 19, 2026) is herein incorporated by reference in its entirety.

### TECHNICAL FIELD

The present invention belongs to the technical field of nanobodies, and more particularly, relates to an anti-ouabain nanobody and an application thereof.

### BACKGROUND ART

Ouabain is a type of cardiotonic steroid compounds, which is widely found in digitalis plants and has high medicinal value. In 1785, Wellington, a British folk physician, used a steroidal plant drug digitalis decoction to treat edema, which had a remarkable effect and attracted wide attention in the medical community. In the middle of the last century, pharmaceutical enterprises in Europe and America competed to develop cardiotonic steroid drugs, among which the most famous is a digoxin preparation, which had unique curative effects on a variety of cardiovascular diseases, and was still an important drug for the treatment of clinical congestive heart failure and other heart diseases for decades. As a cardiotonic steroid compound, ouabain can also be used for the treatment of heart failure and arrhythmia. In terms of a biological mechanism, ouabain mainly acts by inhibiting Na⁺/K⁺-ATPase on a cell membrane. The inhibition of Na⁺/K⁺-ATPase by ouabain increases the intracellular Na⁺ level, and the increase in the Na⁺ level inhibits the activity of a Na⁺/Ca²⁺ exchanger, such that an intracellular Ca²⁺ concentration increases, and a Ca²⁺-related signaling pathway is activated to play a role in enhancing myocardial contractility and treating heart failure. At the end of the 20th century, Hamlyn J M from the United States published a research paper in the journal Nature, proving that endogenous ouabain existed in the human body. Researchers detected and purified this endogenous substance by mass spectrometry in 300 L of human plasma. Hamlyn J M also found that a chemical structure of endogenous ouabain was similar to a structure of plant-derived ouabain, and that they were secreted by the adrenal glands, entered metabolic pathways, and play an important role in regulating arterial pressure, as well as cardiac and renal functions. Further studies found that ouabain was closely related to the occurrence and progression of diseases such as immune disorders and tumors, suggesting that there may be new biological functions besides cardiovascular system. To confirm this hypothesis, it is necessary to accurately quantify ouabain in vivo, and to develop novel antibodies that can neutralize endogenous ouabain.

Since the advent of a hybridoma technology in 1975, monoclonal antibody drugs have been widely applied in the diagnosis and treatment of various diseases. This technology has made important progress in the fields of tumor treatment and diagnosis, molecular detection, etc. In terms of tumor treatment, traditional monoclonal antibodies can target tumors and kill cells, and even lead to necroptosis of tumor cells. In terms of diagnosis, antibody-labeled radioisotopes or fluoresceins play an important role in PET detection and disease imaging. Monoclonal antibodies have the advantages of target specificity, mature preparation process, easy labeling, etc. However, as the application fields expand, the drawbacks of monoclonal antibodies become increasingly obvious. Firstly, the molecular weight of monoclonal antibodies is relatively large, which makes them unstable both in vivo and in vitro. Secondly, monoclonal antibodies have strong immunogenicity and may cause relatively serious side-effects in vivo. In addition, when monoclonal antibodies are applied in molecular imaging, a signal-to-background ratio is too high, which limits their applications in clinical diagnosis of diseases. Therefore, development of novel antibodies is needed, the most representative of which are small molecule antibodies, mainly including Fab fragments, single-chain antibodies, nanobodies, etc.

Nanobodies (Nb) are a new type of small molecule antibodies. They were initially discovered by Hamers Casterman, et al. in 1993 from variable region sequences of heavy-chain antibodies in camels. Further studies have found that two types of antibodies with different structures are naturally present in camel serum. One antibody is a conventional tetramer antibody with two heavy chains and two light chains, while the other is a heavy-chain antibody with only a heavy chain in the absence of two light chains. A variable region of the heavy-chain antibody constitutes a single-chain antibody, which has no light chain and typical constant heavy-chain CH1 domain, and has a relative molecular weight of approximately 15 kDa, which is 1/10 of that of conventional antibodies, and a molecular volume of approximately 4.8 nm*2.2 nm. This is a special and naturally occurring small molecule antibody that is the smallest biologically active functional fragment ever discovered. In camels or sharks, these heavy chain antibodies are biologically active as they can recognize antigens through only a single variable region. Therefore, Nb is the smallest complete antigen-binding domain. Such novel antibodies are present in the bodies of animals such as camels and cartilaginous fish, have structures and physiological functions similar to those of monoclonal antibodies, and thus have great application prospects. Compared with monoclonal antibodies and even other antibodies, Nb has unique advantages, such as high affinity, good stability, and easier soluble expression in microorganisms, greatly reduces the production time and cost, and has a broad application in the fields of accurate diagnosis of various diseases, targeted therapy, and food safety monitoring.

This invention aims to provide materials for constructing lightweight, high-affinity ouabain nanobodies (Nb), and for establishing a thermally stable and highly sensitive ouabain detection method; it also provides research tools for further exploring the novel biological functions of endogenous ouabain.

### SUMMARY OF THE INVENTION

An object of the present invention is to develop novel ouabain Nb with small molecular weight, strong binding capability and stable properties using a genetic engineering method, and to detect the ouabain content by ELISA based on the ouabain Nb, so as to provide a tool for related researches.

In order to fulfill said object, the present invention is implemented by the following solution.

The present invention provides an anti-ouabain nanobody, wherein an amino acid sequence of the nanobody is shown as SEQ ID NO. 1:

EVQLLQSGGLAQPGGSLRLTCTASIGAVYVMGWYRQPPGKQRELVASITS
AGITNYTDSVKSRFIISRDNTKNTVYLQMNSLKPEDTAVYYCNAPLRGRD
DYGGFDWGQGTQVTVSS.

In SEQ ID NO. 1, 1-23 refer to an amino acid sequence of a frame region FR1 of the nanobody: EVQLLQSGGLAQPGGSLRLTCTA (SEQ ID NO. 3); 24-40 refer to an amino acid sequence of a frame region FR2 of the nanobody: MGWYRQPPGKQRELVAS (SEQ ID NO. 4); 41-78 refer to an amino acid sequence of a frame region FR3 of the nanobody: NYTDSVKSRFIISRDNTKNTVYLQMNSLKPEDTAVYYC (SEQ ID NO. 5); 79-89 refer to an amino acid sequence of a frame region FR4 of the nanobody: WGQGTQVTVSS (SEQ ID NO. 6); 90-96 refer to an amino acid sequence of a complementary determining region CDR1 of the nanobody: SIGAVYV (SEQ ID NO. 7); 97-103 refer to an amino acid sequence of a complementary determining region CDR2 of the nanobody: ITSAGIT (SEQ ID NO. 8); and 104-117 refer to an amino acid sequence of a complementary determining region CDR3 of the nanobody: NAPLRGRDDYGGFD (SEQ ID NO. 9).

The present invention further provides a coding gene for the nanobody, wherein a nucleotide sequence of the gene is shown as SEQ ID NO. 2:

GAAGTTCAGCTGCTGCAATCCGGTGGTCTGGCACAACCGGGTGGTTCCC
TGCGCCTGACCTGTACGGCTTCTATTGGTGCGGTTTACGTTATGGGTTG
GTATCGCCAACCACCTGGCAAACAGCGTGAACTGGTTGCATCCATCACC
AGCGCGGGTATTACTAATTATACCGACAGCGTGAAGTCTCGTTTCATCA
TCAGCCGCGATAATACTAAAAACACCGTTTACCTGCAGATGAACAGCCT
GAAACCTGAAGATACGGCGGTTTATTATTGTAACGCTCCGCTGCGTGGT
CGTGACGATTATGGTGGTTTCGATTGGGGTCAAGGTACTCAAGTTACTG
TAAGCAGC.

The present invention provides a recombinant plasmid for a gene containing the anti-ouabain nanobody.

The present invention provides an expression host cell containing the recombinant plasmid.

The present invention further provides a method for preparing the anti-ouabain nanobody.

The preparation method includes the following steps: transforming the recombinant plasmid into a BL-21 genetically engineered bacterium; inducing and then performing preliminary purification by using His-tag Purification resin; and then further refining by means of AKTA ion exchange.

The present invention provides an application of the anti-ouabain nanobody in the preparation of a preparation for detecting ouabain by using an ELISA method. The present invention provides a kit for detecting the ouabain. The kit detects the ouabain based on a competitive ELISA method and uses the nanobody as shown in SEQ ID NO. 1 as a detection antibody.

The present invention provides a method for detecting ouabain. The method includes the following steps:
(1) optimization of antibody concentration: a 96-well adsorptive ELISA plate is coated overnight at 4° C. with 100 μL of ouabain-OVA (20 ng/mL). After discarding the coating solution, the wells are washed five times with PBST. Next, 100 μL of OVA is added for blocking at room temperature for 1 h. After washing the wells five times with PBST, 100 μL of biotinylated ouabain nanobodies at various concentrations is added and incubated at room temperature for 2 h. The wells are then washed again five times with PBST. Subsequently, 100 μL of streptavidin-HRP is added and incubated at room temperature for 1 h, followed by five additional PBST washes. After that, 100 μL of TMB substrate solution is added and the plate was incubated at 37° C. for 10 min. The reaction is terminated by adding 50 μL of stop solution. The absorbance at 450 nm is measured using a microplate reader. The optimal antibody concentration is selected for subsequent detection experiments.
(2) competitive ELISA detection method: a high-adsorption 96-well ELISA plate is coated overnight at 4° C. with 100 μL of ouabain-OVA (20 ng/mL). After discarding the liquid, the wells are washed five times with PBST. Next, 100 μL of OVA is added to each well for blocking at room temperature for 1 hour. The wells are then washed five times with PBST. For the standard group, 100 μL of ouabain standard solution at concentrations of 20 ng/mL, 10 ng/mL, 5 ng/mL, 2.5 ng/mL, 1.25 ng/mL, and 0.6 ng/mL are added to the corresponding wells. For the blank control group (B0), 100 μL of PBS is added. For the detection group, 100 μL of the serum sample to be tested is added. Subsequently, FITC-labeled ouabain nanobody is added to each well, and the plate is incubated at room temperature for 2 hours. After incubation, the liquid is discarded and the wells are washed five times with PBST. Then, 100 μL of streptavidin-RP is added to each well and incubated at room temperature for 1 hour. The wells are washed again five times with PBST. Next, 100 μL of TMB substrate solution is added, and the plate is incubated at 37° C. for 10 minutes, after which 50 μL of stop solution is added to each well. The absorbance at 450 nm is measured using a microplate reader. The mean absorbance (B) for each sample and standard is divided by the absorbance of the blank control (B0) and multiplied by 100% to obtain the percentage absorbance. A standard curve is plotted with the logarithm of the ouabain concentration on the X-axis and the percentage absorbance on the Y-axis. The ouabain concentration (C) in each sample is determined by locating the corresponding percentage absorbance value on the standard curve, reading the abscissa (logarithmic concentration), and calculating the antilogarithm.

The present invention provides an application in which an ouabain nanobody can be used to significantly exert the functions of neutralizing and antagonizing the pharmacological activity of ouabain in vitro.

The present invention discloses the following beneficial effects.

According to the present invention, an antigen is prepared to immunize alpacas and an immune response is monitored, lymphocytes of the immunized alpacas are separated and purified, and their total RNAs are extracted. A phage library with a large output capacity is constructed on the basis of a phage display technology, and the library is subjected to DNA sequencing and database analysis, achieving good diversity and insertion rate, which meet the requirements of screening nanobodies. The library is enriched by antigen ouabain-OVA, and positive clones in the library are effectively enriched after three rounds of solid-phase biopanning. A plurality of clone sequences are obtained by multiple rounds of screening with an indirect ELISA method, and have a strong signal response with the ouabain antigen. The screened clone sequences are expressed and purified to obtain a high-purity antibody protein, and the binding capability of the antibody protein to ouabain is determined by MST. A high-affinity ouabain nanobody is screened, and its biological activity in vitro is verified. The results show that the ouabain nanobody of the present invention can exert the functions of neutralizing and antagonizing the pharmacological activity of the ouabain.

Compared with the prior art, the present invention has the following beneficial effects.

The nanobody obtained in the present invention has the advantages of small molecular weight, high affinity, high expression yield at low production cost, etc. The nanobody can be applied to the detection of the content of ouabain in biological samples, as well as acts as a neutralizing antibody to antagonize the pharmacological activity of the ouabain. In addition, the method for preparing a small molecule nanobody in the present invention has universality and can be applied to the screening and preparation of nanobodies targeting other cardiotonic substances, thus having high application value.

### BRIEF DESCRIPTION OF THE DRAWINGS

To describe the technical solutions in the examples of the present invention or in the prior art more clearly, the following briefly introduces the accompanying drawings required for describing the examples. Apparently, the accompanying drawings in the following description show merely some examples of the present invention, and a person of ordinary skill in the art may still derive other drawings from these accompanying drawings without paying creative efforts.

FIG. 1 shows SDS-PAGE electrophoresis analysis for an ouabain and OVA conjugated product.

FIG. 2 shows serum titer detection for immunized alpacas.

FIG. 3 shows agarose gel analysis for a nested PCR amplification product of a VHH fragment. The left diagram shows Lane 1: a first round of PCR amplification product; and the right diagram shows Lane 1: a second round of PCR amplification product.

FIG. 4 shows agarose gel analysis for products used for a phage library construction. The left diagram shows agarose gel analysis for vector enzyme-digestion products at 15, 30, 60, and 120 min at 50° C. The right diagram shows Lanes 1-3: a gel extracted enzyme-digestion product, a vector digestion product, and a negative control.

FIG. 5 shows library colony capacity estimation for 100 μL of colonies (colonies represent independent clones) diluted from an original library in 10⁴-fold, 10⁵-fold, and 10⁶-fold.

FIG. 6 shows library diversity and sequence characteristic analysis, i.e., analysis for library diversity by random cloning of amino acid sequences from the phage library and analysis for characteristic sequences in an IMGT database.

FIG. 7 shows ELISA assay for an enrichment effect of the phage library.

FIG. 8 shows ELISA screening for positive clones among monoclonal expression products. Indirect ELISA is used for the analysis of monoclonal expression products. Positive clones and negative clones are selected with a value of 0.75, which is more than 5 times of the blank control, as a dividing line.

FIG. 9 shows prokaryotic protein expression and purification of nanobodies. FT refers to a flow-through fluid, and Lanes 1-8 corresponds to batches in collection of components by an ion exchange column.

FIG. 10 shows MST assay for the affinity of ouabain to nanobodies.

FIG. 11 shows the reduction of ouabain-induced cytotoxicity by ouabain nanobodies. The protective effects of ouabain nanobodies at different concentrations on cell survival after treatment with 1 M and 2 M ouabain for 24 h and 48 h are shown.

FIG. 12 shows that the ouabain nanobodies antagonize the increase in Ca²⁺ concentration caused by ouabain. The results in flow cytometry displacement and quantification diagrams of cellular Ca²⁺ indicate that the concentrations of cellular Ca²⁺ treated in an ouabain nanobody group and an ouabain incubation group are significantly inhibited. The experiment is independently repeated for three times, *P<0.05; #P<0.05, which are statistically significant.

FIG. 13 shows a competitive binding ELISA standard curve established on the basis of ouabain nanobodies.

### DETAILED DESCRIPTION OF THE INVENTION

The present invention is further described below in conjunction with the examples, but the description of the examples does not impose any limitation on the protection scope of the present invention.

Unless otherwise defined, all technical and scientific terms used herein have the same meaning as commonly understood by a person of ordinary skill in the art. The terms used in the specification of the present invention herein are for the purpose of describing specific examples only and are not intended to limit the present invention.

The materials or instruments used in the following examples, unless otherwise specified, may be obtained from conventional commercial sources.

### Example 1 Antigen Preparation and Alpaca Immunization

### 1.1 Experimental Materials and Animals

Ouabain was purchased from MCE Company; BSA Protein was purchased from Sigma Aldrich, USA; Coomassie Brilliant Blue Staining Solution was purchased from Beyotime Biotechnology; Carbodiimide and OVA Protein were purchased from Sigma Aldrich, USA; Lymphocyte Separating Solution was purchased from Yeasen Biotechnology; a dialysis bag was purchased from Sangon Biotech (Shanghai) Co., Ltd.; and alpacas were bred by Shenzhen AlpalifeBio, numbered as 1607.

### 1.2 Experimental Method

### 1.2.1 Coupling of Ouabain with Vector Protein OVA

In the present invention, an ouabain compound was coupled with an OVA protein vector by using a carbodiimide method, and 5 mg of ouabain and 5 mg of BSA were dissolved in 0.5 mL of TE buffer; a carbodiimide solution was added dropwise to ouabain and BSA while shaking at 37° C. and reacted in a shaker for 2 h; the solution was put into a dialysis bag; after three days of dialysis, a small amount of dialysis product was extracted and a coupling product was detected by SDS-PAGE; and the rest was frozen in a −20° C. refrigerator for subsequent use.

### 1.2.2 SDS-PAGE and Coomassie Brilliant Blue Staining

SDS-PAGE gel was prepared according to components in proportions in a formula table, and protein separating gel at an appropriate concentration (8%-15%) was prepared according to a molecular weight of a target protein; after lower gel was solidified completely, stacking gel at a concentration of 5% was prepared and inserted with a 10 or 15-well gel comb for immobilization; after protein electrophoresis of the gel, the stacking gel was cut off and slightly moistened with deionized water; the liquid was poured out carefully, and approximately 20 mL of fast Coomassie brilliant blue staining solution was added and shaken in a horizontal shaker at a constant speed, followed by staining for 30 min; and finally, the Coomassie brilliant blue staining solution was recovered, and a gel block was destained and rinsed with deionized water while shaking on the shaker. Every 10 min, fresh deionized water was replaced for rinsing three times, followed by destaining for 2 h.

### 1.2.3 Antigen Immunization for Alpaca

In the present invention, an antigen-BSA conjugate was prepared and used to immunize a naive alpaca, and an animal immunization protocol was shown in the table below.

| Number of days | Immunization method |
|---|---|
| Day 0 | The antigen (1 mg) was mixed with a complete Freund's adjuvant at a ratio of |
| | 1:1, emulsified and then injected subcutaneously |
| Day 14 | The antigen (0.5 mg) was mixed with an incomplete Freund's adjuvant at a |
| | ratio of 1:1, emulsified and then injected subcutaneously |
| Day 28 | The antigen (0.5 mg) was mixed with the incomplete Freund's adjuvant at a |
| | ratio of 1:1, emulsified and then injected subcutaneously |
| Day 42 | The antigen (0.5 mg) was mixed with the incomplete Freund's adjuvant at a |
| | ratio of 1:1, emulsified and then injected subcutaneously |
| Day 56 | The antigen (0.5 mg) was mixed with the incomplete Freund's adjuvant at a |
| | ratio of 1:1, emulsified and then injected subcutaneously |
| Day 70 | The antigen (0.5 mg) was mixed with the incomplete Freund's adjuvant at a |
| | ratio of 1:1, emulsified and then injected subcutaneously |
| Day 80 | 50 mL of peripheral blood was collected for library construction. |

Blood was collected after each alpaca immunization and an immune response was monitored. A total of six rounds of immunization were performed, and 50 mL of peripheral blood was collected from the immunized alpaca 10 days after the last antigen injection.

### 1.2.4 Separation of Mononuclear Lymphoblast B Cells from Peripheral Blood of Alpaca

The collected peripheral blood was mixed with PBS in a volume ratio of 1:2 and mixed well by blowing and sucking with a Pasteur pipette. An appropriate amount of lymphocyte separating solution was added to a 50 mL centrifuge tube, and a peripheral blood diluent was slowly added to the centrifuge tube at 25° C. and blown evenly, and centrifuged at 1000 g for 10 min. Lymphocytes were pipetted, each five tubes of cell suspensions were added into one 50 mL centrifuge tube, and then supplemented to 50 mL with PBS. The centrifuge tube was inverted for mixing well, and then centrifuged at 1000 g and 25° C. for 10 min, and a supernatant was discarded. Pre-cooled PBS was added into the centrifuge tube, lymphocytes were resuspended by blowing and sucking, and then centrifuged at 4° C. and 2500 g for 10 min, and a supernatant was discarded. The resuspended lymphocytes were blown and sucked with PBS, followed by cell counting; and a suspension concentration was adjusted to 5×10⁶ cells/mL. The cell suspension was divided into 1.5 mL RNase-Free centrifuge tubes in a volume of 1 mL per tube which were then centrifuged at 1000 g and 4° C. for 10 min, and a supernatant was discarded; and peripheral blood monocytes were extracted for subsequent experiments.

### 1.3 Experimental Results

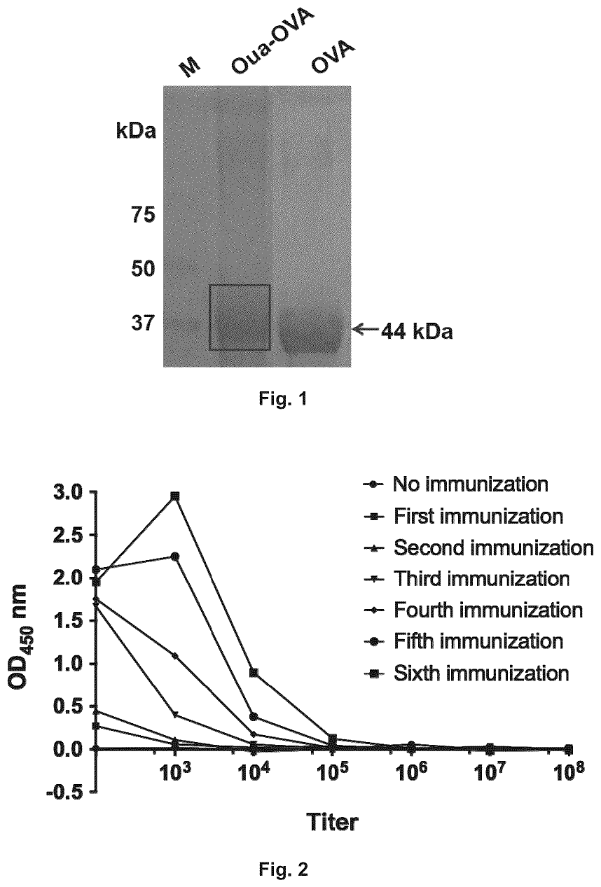

Ouabain was coupled with OVA using a carbodiimide method. Subsequently, an OVA and ouabain-OVA conjugate was subjected to SDS-PAGE electrophoresis analysis. As shown in FIG. 1, a control sample OVA had a significant band around 44 kDa, which was consistent with a theoretical molecular weight (44 kDa) of OVA. Compared with the control sample OVA, the ouabain-OVA conjugate had obvious lagging bands around the molecular weight of 44 kDa. In addition, there were obvious multi-banded products above the molecular weight of 44 kDa, so it was preliminarily determined that the ouabain-OVA conjugate was successfully prepared. The serum titer of the immunized alpaca was detected by an indirect ELISA method. As shown in FIG. 2, compared to pre-immune alpacas, the antigen serum titer of the alpaca increased to 10³-fold after the first immunization, reached 10⁴-fold after the third immunization, and reached 10⁵-fold after the fifth immunization. This result indicated that the antigen produced a strong immune response in the body of the alpaca.

### Example 2: Construction and Screening of Nanobody Phage Display Library

### 2.1 Experimental Materials

A helper phage VCSM13 was purchased from Bio-Viewshine (Beijing) Food Safety Biological Company; Sif I Enzyme was purchased from WISENT Biotech; a T4 DNA ligase and a restriction enzyme were purchased from Thermo Fisher, USA; a DNA gel extraction kit was purchased from Shanghai Sangon Bioengineering Company; a Fast pfu high-fidelity amplification enzyme was purchased from Beijing TransGen Biotech; PCR amplification premix was purchased from Tsingke Biotech Co., Ltd.; DNA Marker was purchased from General Biosystems, Inc.; and a cDNA reverse transcription kit was purchased from Toyobo in Japan.

### 2.2 Experimental Method

### 2.2.1 Synthesis of cDNA and Nested PCR Amplification

Taking RNA as a template, a cDNA pool was obtained by reverse transcription. The amount of RNA used in a reverse transcription 10 L system was 500 ng, and the reverse transcription system was prepared as shown in the table below:

| Components | Volume (μL) |
|---|---|
| 2 × RT mix | 5 |
| Total RNA | 500 ng |
| DEPC water | Up to 10 |

Taking a first round of cDNA as a template, a large number of heavy-chain variable region VHH genes were amplified by nested PCR, and a PCR system was prepared as shown in the table below:

| Components | Volume (μL) |
|---|---|
| 2 × PCR Mix | 25 |
| Phage cDNA | 2 |
| VHH-Primer-F1 | 2.5 |
| VHH-Primer-R1 | 2.5 |
| DEPC water | 18 |

The system was prepared and then amplified in a PCR instrument, and a reaction procedure was shown as follows:

| Number of cycles | Temperature (° C.) | Time |
|---|---|---|
| 1 | 94 | 5 min |
| 2-32 | 94 | 10 s |
| | 65 | 45 s |
| | 72 | 30 s |
| 33 | 72 | 5 min |

Specific amplification primers were as follows:

| Primer name | Sequences (5′→3′) |
|---|---|
| VHH-Primer F1 | GTCCTGGCTGCTCTTCTACAAGG |
| | (SEQ ID NO. 10) |
| VHH-Primer R1 | GGTACGTGCTGTTGAACTGTTCC |
| | (SEQ ID NO. 11) |

In order to increase the capacity of the library, in the present invention, a first round of gel extracted products was used as a PCR template and then subjected to nested PCR amplification. According to conditions shown in the following table, a second round of PCR reaction was carried out:

| Components | Volume (μL) |
|---|---|
| 2 × PCR Mix | 25 |
| First round of extracted products | 2 |
| VHH-Primer-F2 | 2.5 |
| VHH-Primer-R2 | 2.5 |
| DEPC water | 18 |

The system was prepared and then amplified in the PCR instrument, and a second round of PCR reaction procedure was shown as follows:

| Number of cycles | Temperature (° C.) | Time |
|---|---|---|
| 1 | 94 | 5 min |
| 2-32 | 94 | 10 s |
| | 60 | 30 s |
| | 72 | 30 s |
| 33 | 72 | 5 min |

Specific amplification primers were as follows:

| Primer name | Sequences (5′→3′) |
|---|---|
| VHH-Primer- | ACTGGCCCAGGCGGCCGAGGTGCAGCTGSWGSAKTCKG |
| F2222 | (SEQ ID NO. 12) |
| VHH-Primer- | ACTGGCCGGCCTGGCCTGAGGAGACGGTGACCWGGGTC |
| R22 | (SEQ ID NO. 13) |

### 2.2.2 Phage Library Construction

In the present invention, by taking a pComb3xss phage vector as a vector for phage library construction, the vector and a nested PCR product were subjected to enzyme digestion with a restriction enzyme Sfi I first (enzyme-digestion sites were located at 282 bp and 1954 bp of the vector, respectively).

A 20 μL reaction system was prepared as follows:

| Components | Volume (μL) |
|---|---|
| Fast Sfi I | 1 |
| Vector (gene fragment) | 1 |
| 5 × Sfi I Cut buffer | 4 |
| DEPC water | Up to 20 |

The vector and the fragment were subjected to enzyme digestion at 50° C. at the same time for 60 min, followed by agarose electrophoresis. The enzyme-digested vector and fragment were recovered for ligation reaction. In the present invention, T4 ligase was used for ligation, 20 ng of enzyme-digested vector and 8 ng of enzyme-digested insert fragment (a molar ratio of 1:4) were ligated. A ligation system was prepared in the table below:

| Components | Volume (μL) |
|---|---|
| T4 ligase | 1 |
| Vector | 20 |
| Gene fragment | 8 |
| 10 × buffer | 2 |
| DEPC water | Up to 40 |

Subsequently, the enzyme ligation mixture was incubated at 22° C. for 30 min. After ligation, the product was transformed into TG1 competent cells and incubated overnight at 37° C.

### 2.2.3 Phage Hyperinfection Amplification and Titer Determination

In the present invention, a VCSM13 helper phage was used for hyperinfection. All the colonies were scraped away from a library plate and diluted in a 10 mL of 2×TY medium for cryopreservation. 100 μL of bacterial solution was added to a conical flask containing a 2×TY liquid medium, which was then shaken in a constant-temperature shaker until the bacterial growth reached a logarithmic phase with OD₆₀₀ ranging from 0.3 to 0.5. The VCSM13 helper phage was added to the bacterial culture at a volume ratio of 1:100 and incubated at 37° C. for 30 min. The bacterial solution was transferred to a 50 mL centrifuge tube and centrifuged at 2700 rpm and 4° C. for 10 min. A precipitate obtained by centrifugation was resuspended and added to a liquid medium containing 250 mL of 2×TY, 100 mg/mL ampicillin and 50 mg/mL kanamycin. The horizontal shaker was set to 37° C. and 225 rpm, and the culture was shaken overnight for 16 h. The culture was collected and centrifuged at 20,000 g and 4° C. for 30 min. PEG8000/NaCl was added to the supernatant at ¼ volume, inverted for mixing well, and placed in an ice bath for no less than 30 min. Centrifugation was performed at 4000 rpm and 4° C. for 30 min, and a supernatant was discarded. A phage precipitate was resuspended with 1 mL of PBS solution and then placed into a 1.5 mL Eppendorf tube, and centrifuged at 20,000 g and 4° C. for 1 min. A supernatant obtained by centrifugation was collected. A small amount of supernatant was taken for the determination of phage titer, and the rest was frozen in an −80° C. refrigerator.

In the present invention, the phage titer was determined by a double-layer agar method. The phage was diluted to 10⁻¹ to 10⁻¹⁰ pfu with an LB medium; 1 μL of phage in each dilution was mixed well in 100 μL of TG1 bacterial solution, allowed to stand at 37° C. for 20 min, then added with 3 mL of top agar, inverted for mixing well and then poured into an LB solid medium plate, and cultured overnight at 37° C. Plaques were counted and phage titers were calculated.

### 2.2.4 Phage Library Biopanning

The library was biopanned through cycles of binding, washing, elution, and amplification. 20×ELISA coating buffer from the biotechnology company was pre-mixed with ouabain-OVA, and added to a 96-well ELISA plate at 100 μL/well. The concentrations in three rounds of coating were 30 μg/mL, 15 μg/mL, and 7 μg/mL in sequence. The plates were coated overnight at 4° C., followed by five washes with PBST. A 5% BSA solution was prepared in advance, added to each well at 100 μL for blocking, and the plate was washed five times with PBST. A phage with a phage titer of 10⁻¹¹ pfu (phage-to-bacteria ratio of 50:1) was prepared and added to the plate at 100 μL/well, incubated in a horizontal shaker for 2 h, and washed with PBST for five times. Liquid in the plate was discarded. Glycine was added at 100 μL/well to perform elution for 15 min, Tris-HCl was added to neutralize the pH, completing the elution process. The eluent was recovered, and a phage with a titer of 10⁻¹¹ pfu was prepared to infect TG1 Escherichia coli. The titer was detected and an enrichment coefficient was calculated. The infected bacteria were coated to a flat plate, all colonies were collected, and whether to carry out the next round of screening was determined according to an enrichment titer.

### 2.2.5 Screening of Positive Clones from Phage Library

In the present invention, positive clones were screened in an enrichment library by an indirect ELISA method, and hyperinfected bacteria-plate clones after three rounds of enrichment were picked and added to a 96-well plate, infected with a VCSM13 helper phage for 14 h, and centrifuged. 100 μL of supernatant was transferred to a 96-well plate pre-coated with 5 μg/mL ouabain-OVA, and incubated at 37° C. for 45 min. The plate was washed with PBST for six times, added with a 1:5000 dilution of HRP-labeled anti-M13 monoclonal antibody at 100 μL/well, and incubated in a horizontal shaker for 45 min at room temperature. The plate was washed with PBST for three times, added with a TMB color-developing solution at 100 μL/well, and incubated at 37° C. for 10 min. When the solution gradually turned blue and showed obvious shade changes, 2 mM dilute sulfuric acid was added at 50 μL/well to terminate the color-developing reaction, and an OD₄₅₀ value was read by a microplate reader immediately. Wells with an OD₄₅₀ value five times higher than that of a negative control were sequenced to obtain a final nanobody sequence, which was coded for an ouabain nanobody.

### 2.3 Experimental Results

### 2.3.1 Nested PCR Amplification of VHH Fragment

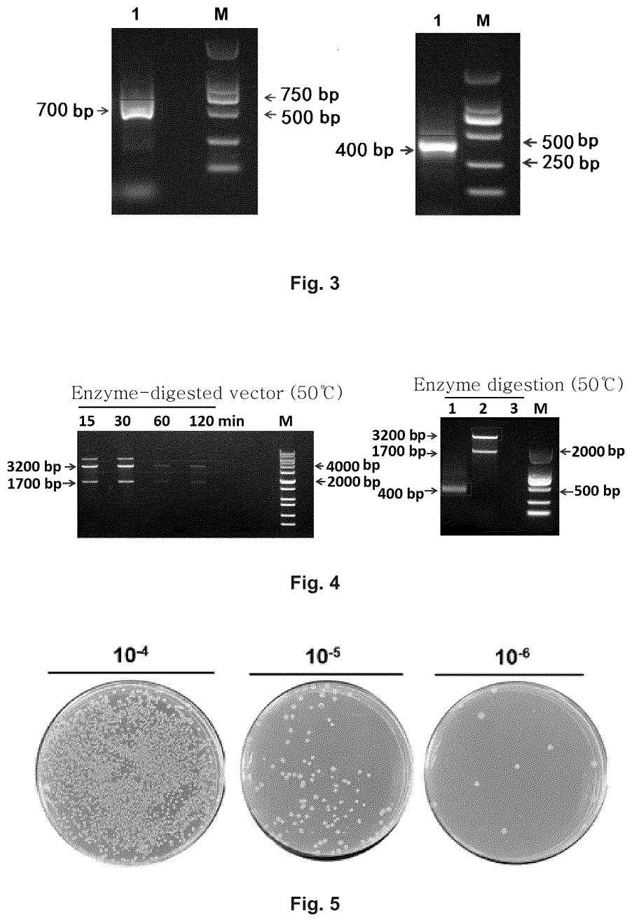

In the present invention, a VHH gene was amplified by nested PCR. As shown in FIG. 3, total RNA extracted above was reversely transcribed to obtain cDNA. The VHH gene fragment was subjected to two rounds of PCR amplification with two pairs of specific primers. The first round of PCR amplification product was subjected to agarose gel electrophoresis analysis. Lane 1 showed a DNA band of approximately 700 bp. Gel purification was carried out to obtain a target band as a template for the next round of PCR. The second round of PCR product was subjected to agarose gel electrophoresis analysis to obtain a DNA band of approximately 400 bp in size, and the gel purification was carried out to obtain a target band product for subsequent construction of a recombinant plasmid. Two rounds of PCR amplification products had bands in size consistent with that of an expected DNA fragment, and could be used for phage library construction.

### 2.3.2 Phage Library Construction

As shown in FIG. 4, library construction requires to perform enzyme digestion on the amplified VHH fragment product and a phage vector pComb3xss, both of which were subjected to single enzyme-digestion with an Sfi I enzyme. Firstly, conditions for the enzyme-digestion efficiency of a vector will be optimized, and enzyme-digestion products of the vector under different conditions were subjected to agarose gel electrophoresis analysis. It could be seen that the enzyme digestion at 50° C. for 60 min showed that clear vector enzyme-digestion bands were 1700 bp and 3200 bp in size respectively, which were consistent with the size of an expected vector enzyme-digestion band, achieving a good enzyme-digestion effect. According to the above enzyme-digestion reaction conditions, the vector and the VHH fragment were enzyme-digested at the same time, and a clear band of the enzyme-digested vector and VHH fragment was displayed on agarose gel, which was consistent with the size of an expected band.

### 2.3.3 Quality Analysis for Phage Library

As shown in FIG. 5, the library was estimated to contain approximately 1×10⁸ colonies by counting clones on a gradient dilution plate. Therefore, the capacity of colonies of the library constructed in the present invention was 1×10⁸ cfu, which was consistent with a standard for screening a nanobody library and could be used for subsequent nanobody screening work.

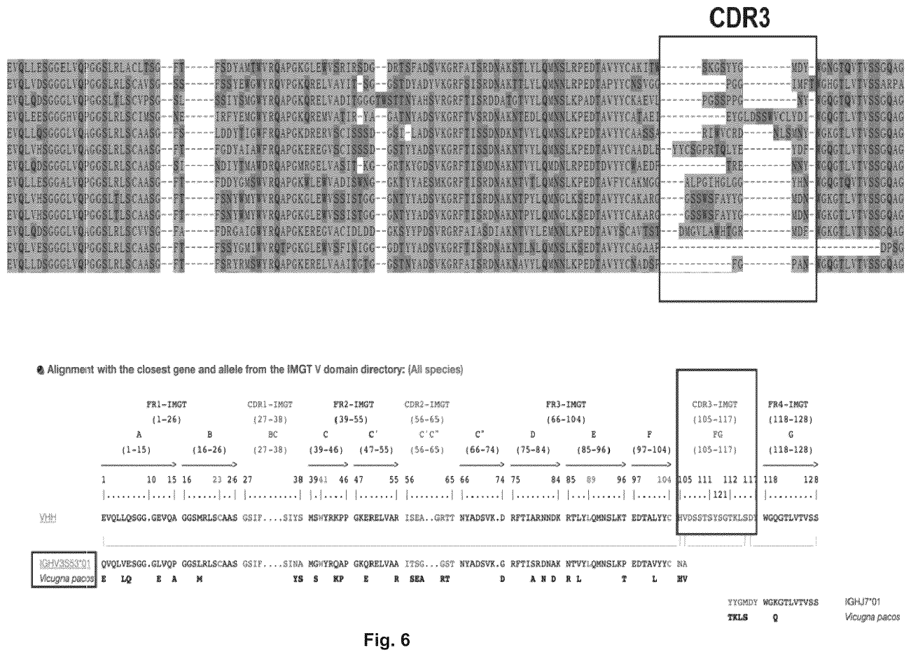

Twenty monoclonal colonies were randomly picked from the gradient dilution plate and aligned with amino acid sequences translated by biological software by means of DNA sequencing. As shown in an upper diagram of FIG. 6, twenty amino acid sequences displayed significant diversity, particularly in the CDR3 regions. The high degree of variation in these antigen-binding regions suggests that they may recognize different epitopes. Therefore, the diversity of the constructed library was preliminarily determined to be 100% in the present invention. Meanwhile, one amino acid sequence was randomly picked and submitted to an IGMT database for analysis in the present invention. As shown in a lower diagram of FIG. 6, analysis revealed that this sequence showed a high degree of homology with a Vicugna IGHV3S53*1 sequence, indicating that this sequence was consistent with basic characteristics of nanobody sequences already available in a database. In summary, the nanobody library constructed in the present invention had a high insertion rate and good diversity, and the nanobody library was successfully constructed and of good quality for subsequent biopanning work.

### Example 3 Biopanning and Expression Application of Nanobodies

### 3.1 Experimental Materials

Glycerin was purchased from Sigma, USA; a DMEM medium, a trypsin cell digestion solution, penicillin and streptomycin were purchased from Wiesent Biotech; a serum-free cell cryopreservation solution was purchased from NCM Biotech; an RP-labeled rabbit secondary antibody and an RP-labeled murine secondary antibody were purchased from China Bioworld; a Trizol reagent was purchased from Shanghai Sangon Bioengineering Company; T4 DNA ligase was purchased from Thermo Fisher, USA; a DNA gel extraction kit was purchased from Shanghai Sangon Bioengineering Company; a Fast Pfu high-fidelity amplification enzyme was purchased from Beijing TransGen Biotech; a restriction enzyme was purchased from Thermo Fisher, USA; RIPA lysate (strong) and RIPA lysate (weak) were purchased from Beyotime Biotechnology; PCR amplification premix, DL5000 DNA Marker, and Agarose were purchased from Tsingke Biotech Co., Ltd.; and a cDNA reverse transcription kit was purchased from Toyobo, Japan.

### 3.2 Experimental Method

### 3.2.1 Phage Library Biopanning

The library was biopanned through cycles of binding, washing, elution, and amplification. 20×ELISA coating buffer from the biotechnology company was pre-mixed with ouabain-OVA, and added to a 96-well ELISA plate at 100 μL/well. The concentrations in three rounds of coating were 30 μg/mL, 15 μg/mL, and 7 μg/mL in sequence. The plates were coated overnight at 4° C., followed by five washes with PBST. A 5% BSA solution was prepared in advance, added to each well at 100 μL for blocking, and the plate was washed five times with PBST. A phage with a phage titer of 10⁻¹¹ pfu (phage-to-bacteria ratio of 50:1) was prepared and added to the plate at 100 μL/well, incubated in a horizontal shaker for 2 h, and washed with PBST for five times. Liquid in the plate was discarded. Glycine was added at 100 μL/well to perform elution for 15 min, Tris-HCl was added to neutralize the pH, completing the elution process. The eluent was recovered, and a phage with a titer of 10⁻¹¹ pfu was prepared to infect TG1 Escherichia coli. The titer was detected and an enrichment coefficient was calculated. The infected bacteria were coated to a flat plate, all colonies were collected, and whether to carry out the next round of screening was determined according to an enrichment titer.

### 3.2.2 Screening of Positive Clones from Phage Library

In the present invention, positive clones were screened in an enrichment library by an indirect ELISA method, and hyperinfected bacteria-plate clones after three rounds of enrichment were picked and added to a 96-well plate, infected with a VCSM13 helper phage for 14 h, and centrifuged. 100 μL of supernatant was transferred to a 96-well plate pre-coated with 5 μg/mL ouabain-OVA, and incubated at 37° C. for 45 min. The plate was washed with PBST for six times, added with a 1:5000 dilution of HRP-labeled anti-M13 monoclonal antibody at 100 μL/well, and incubated in a horizontal shaker for 45 min at room temperature. The plate was washed with PBST for three times, added with a TMB color-developing solution at 100 μL/well, and incubated at 37° C. for 10 min. When the solution gradually turned blue and showed obvious shade changes, 2 mM dilute sulfuric acid was added at 50 μL/well to terminate the color-developing reaction, and an OD₄₅₀ value was read by a microplate reader immediately. Wells with an OD₄₅₀ value five times higher than that of a negative control were sequenced to obtain a final nanobody sequence, which was coded for an ouabain nanobody.

### 3.2.3 Alignment Analysis of Positive Sequences

Positive clones were imported into MEGA software for sequence alignment, and molecular evolutionary tree analysis was performed after alignment. After analysis, candidate nanobody sequences are uploaded to an IMGT database for sequence analysis.

### 3.2.4 Construction of Nanobody Expression Vector

In the present invention, pET-32a was used as an expression vector, BamH I and Xho I were selected as enzyme-digestion sites, and PCR products amplified with positive clones were subjected to agarose electrophoresis. With the positive clones as a template, a VHH fragment was amplified using specific primers.

A PCR amplification system was shown in the table below:

| Components | Volume (μL) |
|---|---|
| 2 × PCR Mix | 25 |
| Phage DNA | 2 |
| Primer-F | 2.5 |
| Primer-R | 2.5 |
| DEPC water | 18 |

The reaction system was shown in the table below:

| Number of cycles | Temperature (° C.) | Time |
|---|---|---|
| 1 | 94 | 5 min |
| 2-32 | 94 | 10 s |
| | 60 | 30 s |
| | 72 | 30 s |
| 33 | 72 | 5 min |

The sequences of specific amplification primers were shown in the table below:

| Primer name | Sequences (5′→3′) |
|---|---|
| Primer-F | ACAGCTATCGCGATTGCAGTG |
| | (SEQ ID NO. 14) |
| Primer-R | AGAAGCGTAGTCCGGAACGT |
| | (SEQ ID NO. 15) |

A pET-32a plasmid and the above gel extracted product were subjected to enzyme digestion with BamH I and Xho I. After enzyme digestion at 37° C. for 30 min, agarose electrophoresis was performed for detection. An enzyme-digested fragment was recovered by gel, and its concentration was determined. An enzyme-digestion system was shown in the table below:

| Components | Volume (μL) |
|---|---|
| Fast BamH I | 1 |
| Fast Xho I | 1 |
| Vector (PCR fragment) | 1 |
| 5 × Cut buffer | 4 |
| DEPC water | Up to 20 |

The enzyme-digested vector and fragment were ligated at a molar ratio of 1:4, and a ligation reaction system was shown in the table below:

| Components | Volume (μL) |
|---|---|
| T4 ligase | 1 |
| Vector | 20 |
| VHH fragment | 8 |
| 10 × buffer | 2 |
| DEPC water | Up to 20 |

After ligation at 22° C. for 30 min, they were transformed to BL21 (DE3) competent cells, and positive clones were taken next day and sequenced.

### 3.2.5 Prokaryotic Expression of Nanobodies

A bacterial solution containing a recombinant plasmid was inoculated into 100 mL of LB medium supplemented with kanamycin at a 1:50 volume ratio and incubated with shaking at 37° C. until OD₆₀₀ was approximately 0.6. IPTG was then added at final concentrations of 0, 0.1, and 10 mM to induce protein expression under various temperature and time conditions. SDS-PAGE analysis was performed to assess protein expression, and the induction conditions were optimized accordingly. Centrifugation was performed at 8000 rpm and 4° C. for 15 min, and a supernatant was discarded. A PBS solution was added to resuspend a bacterial precipitate, a bacterial lysed protein was crushed by ultrasonication, and a specific procedure of an ultrasonic crusher was 3 s of ultrasonication and 9 s of pause, a total of 45 min. The disrupted solution was centrifuged at 13000 rpm and 4° C. for 30 min, a supernatant was collected and placed at 4° C., and a precipitate was frozen at −20° C.

### 3.2.6 Protein Purification of Nanobodies

Every 4 mL of bacterial lysate supernatant was added to 1 mL of uniformly mixed 50% His-tag BeyoGold, and the bacterial lysate supernatant and 50% His-tag BeyoGold were mixed. The mixture was incubated for 60 min at 4° C. with slow shaking. A mixture of lysate and His-tag Purification resin were added to an empty column tube. Under the action of gravity, the liquid in the column was allowed to flow out and collected. 1-2 column volumes of native lysate was added for totally five times, followed by 1-2 column volumes of native wash solution for totally five times. A target protein was eluted for three to five times each time with one column volume of native eluate. Each eluate was collected separately into different Eppendorf tubes. According to the requirements, Milipore protein concentration tubes are adopted. A tube with a 3 kDa molecular weight cutoff was selected to concentrate the sample to an appropriate volume. The sample was added to the concentration tube and centrifuged at 3000 g and 25° C. for 30 min, and a supernatant was collected. Then, the concentration was determined. Depending on the protein purity, a protein was further purified with an AKTA pure protein purifier, and separated and purified using a Superdex 75 gel column or ion exchange column. A buffer system contained 0.02 M Tris-HCl and 1 M NaCl, with pH of 8.0 (balance buffer was adjusted according to an isoelectric point). The system flow rate was set to 0.5-1 mL/min according to an instrument pressure, a chromatographic column was pre-equilibrated and then loaded with a sample using Sample loop. Components flowing at different times were collected using an automated component collector. SDS-PAGE electrophoresis analysis was used to detect different protein components, and the protein components were frozen in an −80° C. refrigerator.

### 3.2.7 Fluorescent Labeling of Protein

In the present invention, the affinity between the antibody protein and the antigen was detected by a MicroScale Thermophoresis (MST) technology, which required at least one of ligand molecules or receptor proteins to be fluorescently labeled. In the present invention, a Monolith RED-NHS next-generation protein labeling kit was selected to label a target antibody.

### (I) Protein Labeling

7 μL of RED-NHS second-generation dye and 7 L of NHS labeling buffer were added into a 1.5 mL Eppendorf tube, and mixed well by blowing and sucking to obtain a dye solution with a final concentration of 300 μM. A new 1.5 mL Eppendorf tube was additionally taken and added with 90 L of protein sample (10 μM). 10 L of the above dye solution was added to the protein sample, mixed well by blowing and sucking to obtain 100 μL of dye protein solution, and incubated at room temperature for 30 min in dark place. For subsequent protein purification, refer to Experimental Method 4.3.3.

### (II) Determination of Degree of Labeling (DOL): The DOL was Calculated According to the Following Formula:

$$c ( M ) = \frac{{A}_{205} - ( {A}_{650} \times 0.19 )}{31 \times M {W}_{p r o t e i n ( D a )}}$$

A calculation formula of DOL was as follows.

$$DOL = \frac{{A}_{650}}{195000 {M}^{- 1} c {m}^{- 1} \times C ( M )}$$

### 3.2.8 MST Assay for Affinity Between Antibody Protein and Ouabain

First, a gradient dilution of the ouabain solution was prepared. APBS solution containing 0.05% Tween-80 was used to dilute ouabain to a final concentration of 1 μM. Then, 10 μL of this 1 μM ouabain solution was transferred into a 0.2 mL PCR tube, labeled as PCR tube No. 1. PCR tubes No. 2 to No. 16 were each preloaded with 10 μL of PBS containing 0.05% Tween-80. A serial (twofold) dilution was performed by transferring 10 L from PCR tube No. 1 to PCR tube No. 2, mixing thoroughly, and then transferring 10 μL from No. 2 to No. 3, and so on, up to tube No. 16. With each transfer and mixing, the ouabain concentration in each subsequent tube was exactly half that of the previous tube. A labeled Nb protein to be detected was diluted to a concentration of 10 nM, and 10 μL of labeled Nb protein solution was added to each PCR tube, and mixed well by blowing and sucking with a pipette. The protein concentration of an antibody to be detected in each PCR tube was 5 nM. An equal volume of labeled antibody protein was added to each PCR tube, and pipetted into a capillary, and the affinity between the antibody and the ligand was detected by MST. A special capillary was used to pipette the liquid in the PCR tube, taking care not to pipette air bubbles. Parameters were set on a machine to detect the affinity between the nanobody to the ouabain.

### 3.3 Experimental Results

### 3.3.1 Biopanning and Enrichment of Phage Library

After the construction of an original library was completed, a total of three rounds of biopanning was performed on a phage library in the present invention, wherein in the first round of panning, a coating antigen ouabain-OVA concentration was 30 μg/mL; in the second round of panning, a coating antigen concentration was 15 μg/mL; and in the third round of panning, the coating antigen concentration was 7 μg/mL. The results were shown in Table 1. Compared with the first round of panning, the number of positive clones was enriched by 1000 fold and the recovery rate was significantly increased in the third round of panning. In addition, an elution ratio of phages was stable in the second and third rounds of panning, which further indicated that the positive clones were significantly enriched.

TABLE 1
Phage enrichment parameters

| Number of rounds | Input (PFU) | Output (PFU) | Recovery rate (%) |
|---|---|---|---|
| 1 | 1 × 10¹¹ | 2 × 10⁵ | 2 × 10⁻⁶ |
| 2 | 1 × 10¹¹ | 1 × 10⁷ | 1 × 10⁻⁴ |
| 3 | 1 × 10¹¹ | 2 × 10⁸ | 2 × 10⁻³ |

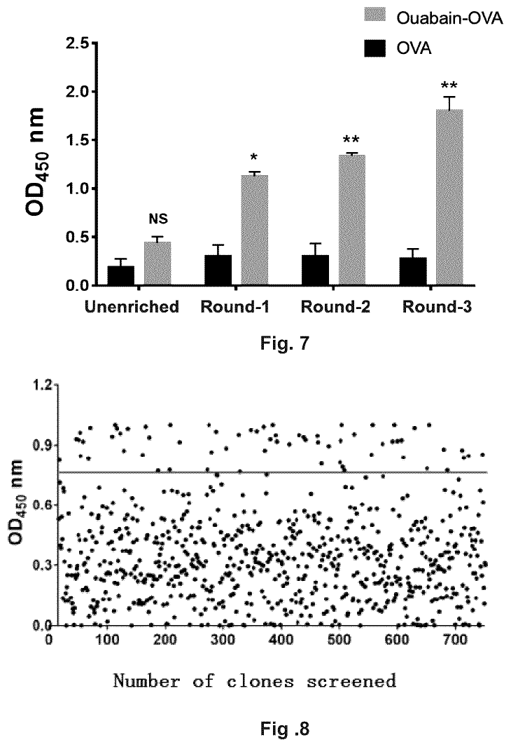

In order to further evaluate a library enrichment effect, the changes in antigen and antibody-binding signals during each round of enrichment and panning were detected by an indirect ELISA method. As shown in FIG. 7, the original library displayed a low binding signal to both OVA and the antigen ouabain-OVA before panning. After three rounds of enrichment, a binding signal of the library to ouabain-OVA was significantly enhanced, which was increased by eightfold compared with a binding signal of the library to OVA, indicating that positive clones that specifically bound to ouabain-OVA in the library were effectively enriched. Based on the comprehensive analysis in terms of the recovery rate of panning and the indirect ELISA detection results, it was indicated that the enrichment effect of the Nb library was good, providing a guarantee for subsequent screening to obtain ouabain Nb.

### 3.3.2 Screening and Identification of Positive Clones

In the present invention, the positive clones were screened by the indirect ELISA method. Monoclones were screened in multiple rounds from an enrichment library, and periplasm expression products of the monoclones were detected by the indirect ELISA method. As shown in FIG. 8, an index with OD₄₅₀ value of more than 5 times of a blank control was selected as a dividing line, and finally a plurality of positive clones were picked for DNA sequencing and database analysis.

### 3.3.3 Prokaryotic Expression of Nanobodies

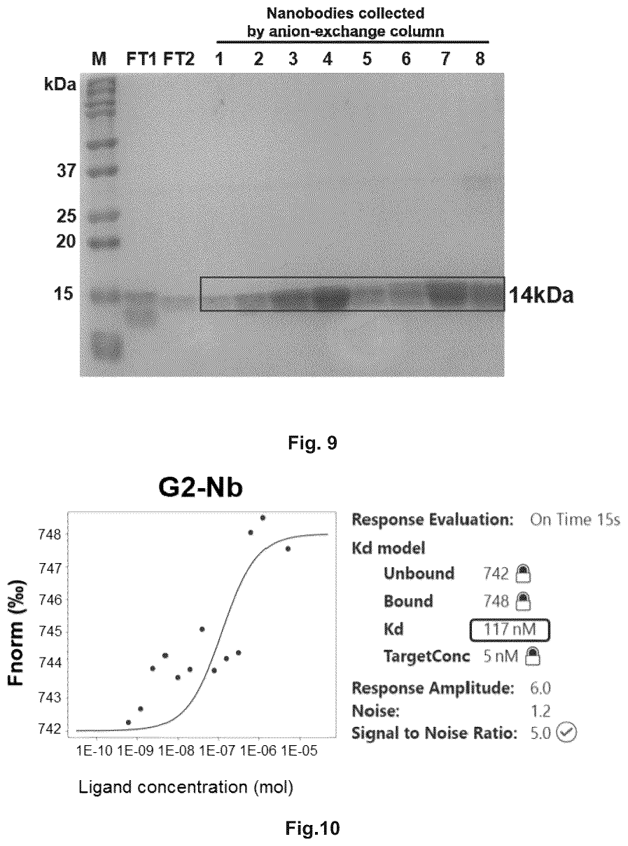

In the present invention, a recombinant plasmid for prokaryotic expression was constructed. After the construction and sequencing verification were completed, the recombinant plasmid was transferred into host bacteria, and induced with 100 mM IPTG at 37° C. for 4 h. After the bacterial solution was ultrasonically crushed, a supernatant was collected and purified by a nickel column, and the purified product was subjected to ion exchange for SDS-PAGE electrophoresis analysis. As shown in FIG. 9, it can be seen that recombinant protein products had obvious bands around 14 kDa, which were consistent with a theoretical molecular weight of the recombinant protein, indicating that the recombinant protein was successfully expressed.

### 3.3.4 MST Assay for Affinity Between Nanobody and Ouabain

In the present invention, an antibody protein was expressed and purified by nickel column affinity chromatography. The antibody protein was labeled with a nucleic acid dye using a MonolithRED-NHS second-generation protein labeling kit. The affinity between a candidate Nb protein and ouabain was determined using MST. As shown in FIG. 10, the affinity determination was rapidly performed using MST by measuring fluorescence changes resulting from the interaction between proteins and small molecules. The results showed that the fluorescent protein was uniformly distributed, indicating effective labeling. The binding ability of the Nb to ouabain was then measured. The amino acid sequence of Nb was shown in SEQ ID NO. 1 and named G2-Nb.

### Example 4 In-Vitro Neutralization of Nanobody for Pharmacological Activity of Ouabain

### 4.1 Experimental Materials

See 3.1 for experimental materials

### 4.2 Experimental Method

### 4.2.1 Cell Culture and Passage

Cells were cultured in a 37° C., 5% CO₂ cell incubator by using a DMEM medium containing 10% of serum and penicillin. When the cell growth density reached 80%-90% in a dish, the cells were washed with a sterile PBS solution, digested with an appropriate volume of trypsin, and placed in the cell incubator and cultured for 2 min. Digestion was terminated by adding fresh medium, and the cell suspension was transferred to a 15 mL centrifuge tube and centrifuged at 800 g for 3 min. A supernatant was removed and added to a new medium. The cells were resuspended by blowing and sucking, and then ⅓ of the suspension was evenly distributed in a culture dish with the new medium, and cultured in the 37° C., 5% CO₂ cell incubator.

### 4.2.2 Extraction of Cell Protein

Cells with good growth status were selected, added with a drug or transfected, and then put into a centrifuge tube. The cells were washed twice with sterile PBS and added with an appropriate amount of RIPA lysate. The cells were fully shaken and mixed, lysed in an ice bath, shaken once at an interval of 10 min, in a total of 3 times, and centrifuged at 12000 rpm for 10 min, and a supernatant was taken for storage.

### 4.2.3 Determination of Protein Concentration by BCA Method

A BCA working solution was mixed with A and B solutions in a volume ratio of 50:1 to prepare 200 μL of working solution for each sample. 2 μL of each sample was taken, filled with PBS up to 10 μL, and mixed well with 200 μL of BCA working solution. The solution was cultured at 37° C. for 30 min, the OD₅₆₀ absorbance of the solution was measured by colorimetric photometry, and the content of the solution was calculated from its standard curve.

### 4.2.4 MTT Determination for Cell Survival Rate of Ouabain

By methyl thiazolyl tetrazolium (MTT), the cell survival rate was measured. When the cell density in a 6-well plate reached 80%-90%, the cells were digested, and centrifuged at 1000 rpm for 5 min; a cell precipitate was left, added with 1 mL of culture solution and then resuspended; and the cells were counted with a cell counting plate. 100 L of cell suspension was added to a 96 ELISA plate at 1×10⁴ cells/well, and sterile PBS was added around cell wells. The cells were cultured on a 96-well plate for 12 to 24 h. When the cell growth density reached 60% to 80%, a drug could be added to treat the cells at this point. MTT was prepared to a solution with a concentration of 5 mg/mL under dark conditions, and 15 L of the solution was added to cell wells and placed in the cell incubator for 4 h. The solution in the 96-well plate was discarded, and added with DMSO at 150 μL/well. The 96-well plate was placed on a horizontal shaker and shaken at room temperature for 10 min. The absorbance of each well was detected with a microplate reader at 490 nm and the cell survival rate was calculated.

A calculation formula for the cell survival rate was as follows:

cell survival rate=[(OD_{experimental group}−OD_{zero treatment group})/(OD_{control group}−OD_{zero treatment group})]*100%.

### 4.2.5 Flow Cytometry Assay for Intracellular Ca²⁺ Level

The cells were cultured in a 12-well cell culture dish. When the cell growth density reached 60% to 80%, a drug could be added to treat the cells. The medium was removed from the treated cells under dark conditions, and the cells were rinsed with pre-warmed HBSS for three times to eliminate esterases and other substances in the medium to reduce their degradation to an ion probe. 1-5 μM of Fluo4-AM working solution was added, such that the cells were completely covered with the working solution and incubated in a cell incubator for 40 min at a constant temperature. The Fluo4-AM working solution was discarded, and the cells were washed with HBSS for three times to thoroughly remove any remaining dye Subsequently. 1 mL of HBSS was added to each dish, and the cells were incubated at room temperature for 20-30 min to ensure that an AM body was completely de-esterified in the cells. The cells were completely digested with trypsin, centrifuged at 2500 rpm for 5 min, washed with HBSS for three times, finally resuspended with 400 μL of HBSS and transferred to a flow cytometer tube. A BL1/FITC channel was selected for determination.

### 4.2.6 Competitive Binding ELISA Assay for Endogenous Ouabain

The specific steps are described in the Summary section.

### 4.3 Experimental Results

### 4.3.1 Reduction of Ouabain-Induced Cytotoxicity by Ouabain Nanobodies

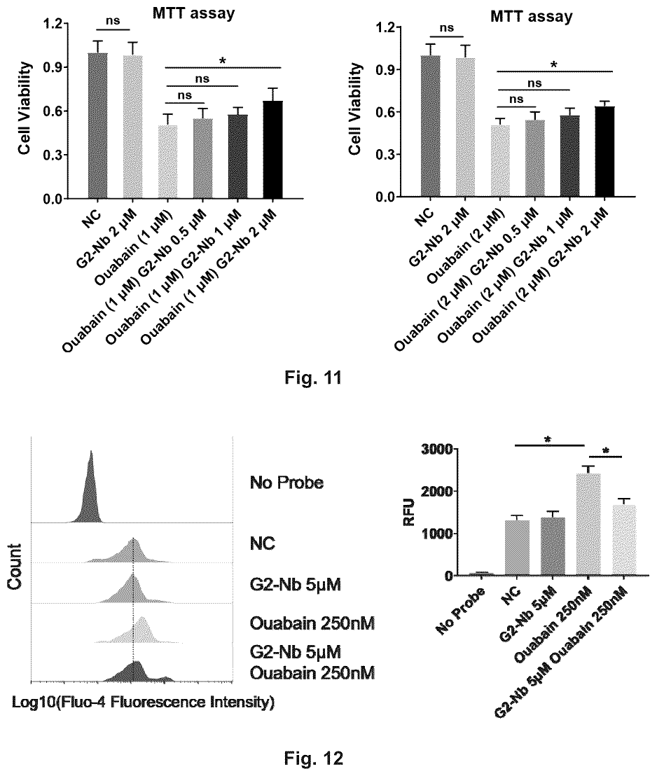

High-concentration ouabain can lead to apoptosis of cells. To study the pharmacological effect of ouabain Nb, this study treated Hela cells with ouabain and ouabain Nb, and detected the effects of ouabain and antibody protein on the cell survival rate by an MTT method. As shown in FIG. 11, the Hela cells were treated with 1 M and 2 M of ouabain for 48 h and 24 h, respectively. At this time, the survival rate of the cells treated with ouabain was reduced to half of the original. Therefore, this ouabain concentration was selected, and the cells were pre-incubated with three concentrations (0.5, 1, 2 μM) of ouabain Nb for 30 min before treatment. The MTT results showed that different concentrations of ouabain Nbs had a certain neutralizing effect, reduce the toxicity of ouabain to cells and increase the survival rate of the HeLa cells compared with a ouabain control group. The above results showed that the ouabain Nb prepared in this study could neutralize a certain concentration of ouabain in vitro and reduce its toxicity. 4.3.2 Antagonization of ouabain nanobody for increase in Ca²⁺ concentration caused by ouabain Ouabain can cause a reduction in intracellular K⁺ and accumulation of Na⁺ by inhibiting Na⁺/K⁺-ATPase. This led to depolarization of a cell membrane, as well as activation of a Ca²⁺ channel and formation of Na⁺—Ca²⁺ exchange, thereby increasing the Ca²⁺ concentration and eventually triggering the release of Ca²⁺ from the endoplasmic reticulum. To further verify the biological activity of ouabain Nb, the Ca²⁺ concentration in the HeLa cells was detected by neutralizing ouabain monomers with ouabain Nb. As shown in FIG. 12, the Ca²⁺ concentration in a 250 nM ouabain group was significantly higher than that in the control group. After incubation of ouabain Nb (5 μM) and ouabain, the cells were treated and the Ca²⁺ concentration was detected. The results revealed that the Ca²⁺ concentration was significantly reduced. The above results showed that the ouabain Nb played a significant role in cells and was able to antagonize the increase in Ca²⁺ concentration caused by ouabain.

### 4.3.3 Competitive Binding ELISA Assay for Endogenous Ouabain

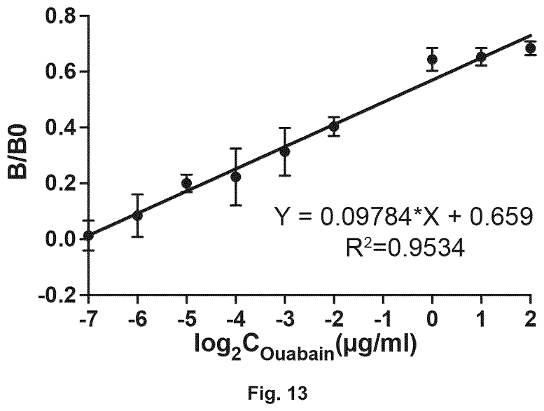

The antigen was immobilized, the working concentration of the antibody was determined to be around 1 μg/mL, and the optimal antibody concentration was selected for subsequent detection experiments. A competitive ELISA standard curve established on the basis of an antibody G2-Nb with an amino acid sequence shown in SEQ ID NO. 1 was shown in FIG. 13 and Table 2. It may be observed that the linear correlation of the curve was good, and the detection range was comprehensively determined to 10-500 ng/mL according to the recovery rate and other parameters. An average OD₄₅₀ value of a standard blank was recorded as B0, the OD₄₅₀ at different drug concentrations and an average value of wells to be detected were recorded as B, and different drug concentrations or a B/B0 ratio of sample wells and a standard deviation of each group of parallel data were calculated. A standard curve was generated by plotting the logarithm of the standard concentration on the x-axis and the B/B₀ ratio on the y-axis. Based on the average absorbance value of the sample wells, the corresponding x-axis value (logarithm of the ouabain concentration) was identified from the curve, and the actual ouabain concentration in the sample was determined by calculating the antilogarithm.

TABLE 2
Recovery rate data of ouabain detection

| | Standard deviation | | Relative |
|---|---|---|---|
| Ouabain | (ng/mL) | Recovery | standard |
| (ng/mL) | (n = 4) | rate (%) | deviation (%) |
| 10 | 19.4 ± 9.7 | 194 | 97 |
| 50 | 43.34 ± 10.2 | 86.7 | 24 |
| 250 | 253.8 ± 31.7 | 101 | 12 |
| 500 | 537.7 ± 41.9 | 109 | 8 |

1. An anti-ouabain nanobody, wherein an amino acid sequence of the anti-ouabain nanobody is as shown in SEQ ID NO. 1.

2. A coding gene for the anti-ouabain nanobody according to claim 1, wherein a nucleotide sequence of the coding gene is as shown in SEQ ID NO. 2.

3. A recombinant plasmid, wherein the recombinant plasmid contains the coding gene for the anti-ouabain nanobody according to claim 1.

4. A method for preparing the anti-ouabain nanobody according to claim 1, comprising the following steps: transforming a recombinant plasmid that contains the coding gene for the anti-ouabain nanobody into a BL-21 genetically engineered bacterium; inducing and then performing preliminary purification by using His-tag Purification resin; and then further purifying by means of AKTA ion exchange.

5. A method of applying the anti-ouabain nanobody according to claim 1 in the preparation of a preparation for detecting ouabain by using an ELISA method.


SEQUENCE LISTING (15 sequences)

SEQ ID NO: 1 - protein, 117 aa, Vicugna pacos
EVQLLQSGGLAQPGGSLRLTCTASIGAVYVMGWYRQPPGKQRELVASITSAGITNYTDSVKSRFIISRDNTKNTVYLQMNSLKPEDTAVYYCNAPLRGRDDYGGFDWGQGTQVTVSS

SEQ ID NO: 2 - DNA, 351 bp, Vicugna pacos
  source 1..351: mol_type = genomic DNA
gaagttcagctgctgcaatccggtggtctggcacaaccgggtggttccctgcgcctgacctgtacggcttctattggtgcggtttacgttatgggttggtatcgccaaccacctggcaaacagcgtgaactggttgcatccatcaccagcgcgggtattactaattataccgacagcgtgaagtctcgtttcatcatcagccgcgataatactaaaaacaccgtttacctgcagatgaacagcctgaaacctgaagatacggcggtttattattgtaacgctccgctgcgtggtcgtgacgattatggtggtttcgattggggtcaaggtactcaagttactgtaagcagc

SEQ ID NO: 3 - protein, 23 aa, Vicugna pacos
EVQLLQSGGLAQPGGSLRLTCTA

SEQ ID NO: 4 - protein, 17 aa, Vicugna pacos
MGWYRQPPGKQRELVAS

SEQ ID NO: 5 - protein, 38 aa, Vicugna pacos
NYTDSVKSRFIISRDNTKNTVYLQMNSLKPEDTAVYYC

SEQ ID NO: 6 - protein, 11 aa, Vicugna pacos
WGQGTQVTVSS

SEQ ID NO: 7 - protein, 7 aa, Vicugna pacos
SIGAVYV

SEQ ID NO: 8 - protein, 7 aa, Vicugna pacos
ITSAGIT

SEQ ID NO: 9 - protein, 14 aa, Vicugna pacos
NAPLRGRDDYGGFD

SEQ ID NO: 10 - DNA, 23 bp, synthetic construct
  source 1..23: mol_type = other DNA
gtcctggctgctcttctacaagg

SEQ ID NO: 11 - DNA, 23 bp, synthetic construct
  source 1..23: mol_type = other DNA
ggtacgtgctgttgaactgttcc

SEQ ID NO: 12 - DNA, 38 bp, synthetic construct
  source 1..38: mol_type = other DNA
actggcccaggcggccgaggtgcagctgswgsaktckg

SEQ ID NO: 13 - DNA, 38 bp, synthetic construct
  source 1..38: mol_type = other DNA
actggccggcctggcctgaggagacggtgaccwgggtc

SEQ ID NO: 14 - DNA, 21 bp, synthetic construct
  source 1..21: mol_type = other DNA
acagctatcgcgattgcagtg

SEQ ID NO: 15 - DNA, 20 bp, synthetic construct
  source 1..20: mol_type = other DNA
agaagcgtagtccggaacgt